(19) United States
(12) Patent Application Publication
(10) Pub. No.: US 2009/0130210 A1
(43) Pub. Date: May 21, 2009

(54) PHARMACEUTICAL COMPOSITIONS OF SIROLIMUS

(76) Inventors: Praveen Raheja, New Delhi (IN); Atul Kaushik, Ghaziabad (IN); Rajesh Gandhi, Gurgaon (IN); Romi Barat Singh, Varanasi (IN); Rajeev Shanker Mathur, Gurgaon (IN)

Correspondence Address: Jayadeep R. Deshmukh, Esq.;Ranbaxy Inc., Suite 2100, 600 College Road East, Princeton, NJ, 08540, US

(21) Appl. No.: 12/127,130

(22) Filed: May 27, 2008

(30) Foreign Application Priority Data
Sep. 11, 2007 (IN) 1920/DEL/2007
May 8, 2008 (IN) 1147/DEL/2008

(51) Int. Cl. A61K 9/36; A61K 31/4355; A61K 9/14

(52) U.S. Cl. 424/479; 514/291; 424/494

(57) ABSTRACT

A pharmaceutical composition of sirolimus comprising an inert core and a sugar overcoat, wherein said sugar overcoat comprises sirolimus, a poloxamer other than poloxamer 188, microcrystalline cellulose and binder has been developed.

### FIELD OF THE INVENTION

The present invention relates to a pharmaceutical composition of sirolimus and process for preparation thereof.

### BACKGROUND OF THE INVENTION

Sirolimus, which is also known as rapamycin, is a macrolide antibiotic produced by Streptomyces hygroscopicus which was first found to have antifungal properties. It adversely affects the growth of fungi such as Candida albicans and Microsporum gypseum. Rapamycin, its preparation and its antibiotic activity were described in U.S. Pat. No. 3,929,992. In 1977, Martel, R. R. et al. reported immunosuppressive properties of rapamycin against experimental allergic encephalitis and adjuvant arthritis in the Canadian Journal of Physiological Pharmacology, (1977) 55, 48-51. In 1989, Calne, R. Y. et al. in Lancet, (1989) No. 2, 227 and Morris, R. E. and Meiser, B. M. in Medicinal Science Research, (1989) No. 17, 609-10, separately reported the effectiveness of rapamycin in inhibiting rejection in vivo in allograft transplantation. U.S. Pat. No. 5,100,899 discloses the use of Rapamycin to inhibit transplantation rejection in mammals.

Its poor oil and water solubility, poses a significant problems in formulating the drug into suitable dosage form. In addition, it has been reported that compositions of sirolimus with conventional excipients show unpredictable dissolution rates, irregular bioavailability profiles, as well as stability problems. Currently, sirolimus is available in two dosage forms namely tablet and oral solution.

U.S. Pat. Nos. 5,989,591 and 5,985,325 disclose a solid dosage unit of rapamycin comprising a core, which is over coated with rapamycin, and a sugar coat containing one or more surface modifying agents, one or more sugars and optionally one or more binders.

U.S. Pat. No. 5,145,684 discloses a nanoparticulate composition comprising particles consisting of a poorly soluble drug having adsorbed onto the surface thereof a non-crosslinked surface stabilizer wherein effective average particle size of drug substance is less than about 400 nm.

### SUMMARY OF THE INVENTION

Now, we have developed an alternate pharmaceutical composition for an oral administration comprising sirolimus and process for preparation thereof.

Hence, according to one of the aspects, there is provided a pharmaceutical composition of sirolimus comprising an inert core and sugar overcoat, wherein the sugar overcoat comprises
a) sirolimus;
b) poloxamer; and
c) a binder selected from the group consisting of hydroxypropylcellulose, hydroxypropyl methyl cellulose or a mixture thereof; and wherein the poloxamer is not poloxamer 188.

In another aspect, there is provided a pharmaceutical composition of sirolimus comprising an inert core and sugar overcoat, wherein said sugar overcoat comprises
a) about 1-5% w/w of sirolimus;
b) about 0.05-5% w/w poloxamer; and
c) about 0.05-4% w/w of a binder selected from the group consisting of hydroxypropylcellulose, hydroxypropyl methyl cellulose or a mixture thereof; and wherein the poloxamer is not poloxamer 188.

In another aspect, there is provided a pharmaceutical composition of sirolimus comprising an inert core and sugar overcoat, wherein said sugar overcoat comprises
a) sirolimus;
b) poloxamer;
c) microcrystalline cellulose; and
d) a binder selected from the group consisting of hydroxypropylcellulose, hydroxypropyl methyl cellulose or a mixture thereof; and wherein the poloxamer is not poloxamer 188.

In another aspect, there is provided a pharmaceutical composition of sirolimus comprising an inert core and sugar overcoat, wherein said sugar overcoat comprises
a) about 1-5% w/w of sirolimus;
b) about 0.05-5% w/w poloxamer;
c) about 0.05-3% w/w of microcrystalline cellulose; and
d) about 0.05-4% w/w of a binder selected from the group consisting of hydroxypropylcellulose, hydroxypropyl methyl cellulose or a mixture thereof; and wherein the poloxamer is not poloxamer 188.

In another aspect, there is provided a pharmaceutical composition of sirolimus comprising an inert core and sugar overcoat, wherein said sugar overcoat comprises
a) about 1-5% w/w of sirolimus;
b) about 0.05-5% w/w poloxamer;
c) about 0.05-0.3% w/w of microcrystalline cellulose;
d) about 0.05-4% w/w of a binder selected from the group consisting of hydroxypropylcellulose, hydroxypropyl methyl cellulose or a mixture thereof; and
e) about 0.02-2% w/w of antioxidant and wherein the poloxamer is not poloxamer 188.

In another aspect, there is provided a process for preparation of pharmaceutical composition of Sirolimus comprising the steps of:
a) Preparation of an inert core;
b) Optionally, seal coating the inert core of step a);
c) Optionally, sub coating the inert core of step b);
d) Preparation of dispersion of sirolimus, poloxamer, microcrystalline cellulose, a binder selected from the group consisting of hydroxypropylcellulose, hydroxypropyl methyl cellulose or a mixture thereof and optionally one or more pharmaceutically acceptable excipients in a suitable vehicle;
e) Coating inert core of step a) or step b) or step c) with sirolimus dispersion of step d); and
f) Optionally, further coating said drug coated cores to obtain desired pharmaceutical composition;

wherein the poloxamer is not poloxamer 188.

In another aspect, there is provided a method of treatment of organ or tissue transplant rejection, autoimmune disease, inflammatory conditions, or multi-drug resistance, the method comprising: orally administering to a subject a pharmaceutical composition of sirolimus comprising an inert core and sugar overcoat, wherein said sugar overcoat comprises
a) sirolimus;
b) poloxamer;
c) microcrystalline cellulose; and
d) a binder selected from the group consisting of hydroxypropylcellulose, hydroxypropyl methyl cellulose or a mixture thereof; and wherein the poloxamer is not poloxamer 188.

### DETAILED DESCRIPTION OF THE INVENTION

“Sirolimus” as employed herein is intended to include amorphous or crystalline form of the drug. The crystalline form may include polymorph form I or II or a mixture thereof.

“Inert core” as used herein includes inert tablet core or inert beads or spheres.

Pharmaceutical composition may be in the form of tablet comprising an inert core and coating of sirolimus dispersion or it may be in the form of capsule comprising coated beads or spheres or granules.

Inert tablet core may be prepared by blending pharmaceutical acceptable excipients such as diluents, glidants and compressing into a suitable size tablet.

Inert tablet core may be further coated with sugar dispersion/solution. Sugar coating may be in the form of seal coating, sub coating, syrup coating and the like.

Inert core is coated with a sugar overcoat comprising sirolimus, poloxamer and hydroxypropyl methylcellulose. Poloxamer act as surfactant and enhances the solubility of poorly soluble drug, sirolimus. Hydroxypropyl methylcellulose is used as a binder in the sugar overcoat.

Poloxamer (polyoxyethylene-polyoxypropylene copolymers) is a crystalline or semi-crystalline material that generally has a molecular weight ranging from about 2000 to about 15,000 daltons. Suitable poloxamers are sold under the trade names PLURONIC and LUTROL, both available from BASF Corporation. Some of the commercially marketed grades of poloxamers not poloxamer 188 include poloxamer 237 (PLURONIC F87), poloxamer 338 (PLURONIC F108), poloxamer 407 (PLURONIC F127). The amount of poloxamer present in the present invention is about 0.05-5 percent by weight of the sugar overcoat, in particular about 0.5-2% by weight of the sugar overcoat. It may be used with other surfactant or surface modifiers selected the group consisting of sorbitan esters, magnesium aluminum silicate, triethanolamine, polyvinyl alcohol, and polyvinylpyrrolidone, polyethoxylated fatty acids, polyethylene glycol 400 distearate, polyethylene glycol-20 dioleate, polyethylene glycol 4-150 mono dilaurate and the like.

Binder is selected from the group consisting of hydroxypropylcellulose, hydroxypropyl methyl cellulose or a mixture thereof. Hydroxypropyl methylcellulose polymers are hydrophilic in nature. It may be used in different viscosity grades such as those available under the brand name Methocel™ available from Dow Chemical Co. and Metolose™ from Shin Etsu Ltd. examples of hydroxypropyl methylcellulose polymers include those available under the brand names Methocel™ E5, Methocel™ E-15 LV and Methocel™ E50 LV.

Hydroxypropylcellulose polymers are hydrophilic in nature. Examples of hydroxypropyl cellulose include those available under the brand names of Klucel GF, Klucel JF, Klucel LF and Klucel EF.

The amount of hydroxypropyl methylcellulose or hydroxy propyl cellulose present in the present invention is from about 0.05% to about 4% by weight of sugar overcoat, in particular 0.2-1.5% w/w.

Microcrystalline cellulose is added in the composition as reinforcing agent. It controls the rate of release of Sirolimus from the sugar coating. The amount of microcrystalline cellulose may vary from about 0.05% to about 3%, in particular, from about 0.05% to about 0.3% w/w based on the weight of sugar overcoat.

The above pharmaceutical composition of sirolimus may contain sirolimus particles having d₉₀ value of from about 2μ to about 10μ and d₅₀ value of from about 0.5μ to about 4μ.

The known particle size analysis methods can be used for determining the particle size, for Example particle size measurement using light, like light-scattering methods, in particular Malvern mastersizer.

Micronization may be carried out using dry milling technique or supercritical fluid technique may be utilized for particle size reduction.

Seal coating is used to prevent moisture penetration into the tablet core and thus prevents the tablet core from disintegrating during the over coating process. Seal coating may comprise shellac, oleic acid, propylene glycol, talc, polyethylene glycol or mixture thereof.

Sub coating as used herein is used to round the edges and build up the tablet size. Sub coating may comprise other excipients selected from the group consisting of starch, talc, calcium carbonate, calcium sulfate or mixtures thereof, in addition to sugar.

Other than sugar coating, tablet may further comprise film coating include functional or non functional layer. The coating may be selected from amongst one or more of those suitable coating materials known in the art. Coating may be performed by applying one or more film forming polymers, with or without other pharmaceutically inert excipients, as a solution/suspension. The tablets may be polished using carnauba wax or any other waxy material, however, the amount of waxy substance selected is optimized so that release of drug from the polymer is not altered due to presence of these excipients. The amount of polishing material may vary from about 0.01 mg/tab to about 1.00 mg/tab, in particular, 0.01-0.04 mg/tab.

Coating is done using any conventional coating technique known in the art, such as spray coating in a conventional coating pan or fluidized bed processor; or dip coating.

The term “pharmaceutically acceptable excipients” as used herein include, sugars, diluents, lubricant/glidants, disintegrating agents, antioxidants and coloring agents.

Sugars may be used to prepare sugar barrier coat or over coat or drug coat wherein the drug coat comprises dispersion of the sirolimus and sugars or one or more pharmaceutical acceptable excipients. Sugar may include lactose, mannitol, sorbitol, sucrose and mixtures thereof.

The term “diluents” as used herein includes calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, sucrose and mixtures thereof.

Specific examples of lubricants/glidants include colloidal silicon dioxide, stearic acid, magnesium stearate, calcium stearate, talc, hydrogenated castor oil, and mixtures thereof.

Disintegrating agents may be selected from starches or modified starches such as starch, modified starch, croscarmellose sodium, crospovidone and sodium starch glycolate.

The composition may further comprise antioxidant, to protect the drug from oxidative degradation. Antioxidants may be selected from group consisting of ascorbic acid, sodium pyrosulphite, glutathion or sorbic acid, tocopherol and the like, in particular tocopherol E-acetate. The amount of antioxidant present in the present invention is from about 0.02% to about 2%, in particular 0.05%-0.5% w/w of sugar overcoat.

Coloring agent may be selected from FDA approved colorants and the examples are Iron oxide, Opalux yellow, Lake of Tartrazine, Allura red, Lake of Quinoline yellow, Lake of Erythrosine.

The vehicle used to prepare the dispersion may be selected from water or its mixture with other organic solvent such as ethanol, methanol, isopropyl alcohol and ether.

According to one of the embodiment, there is provided a process for the preparation of sugar over coated tablet, comprising the steps of:
i) Blending and sifting pharmaceutically acceptable excipients;
ii) Optionally, granulating blend of step i);
iii) Compressing blend of step i) or granules of step ii) into a suitable size tablet to obtain an inert core;
iv) Dissolving/dispersing sirolimus, poloxamer, microcrystalline cellulose, hydroxypropyl methylcellulose and/or hydroxypropyl cellulose and, optionally, one or more pharmaceutically acceptable ingredients in a suitable vehicle;
v) Coating the dispersion of sirolimus of step iv) onto the inert core of step iii);
vi) Optionally, further coating drug coated core of step v).

According to another embodiment, there is provided a process for the preparation of sugar over coated tablet, comprising the steps of:
i) Blending and compressing pharmaceutically acceptable excipients to obtain an inert core;
ii) Coating a dispersion or solution of shellac and optionally one or more pharmaceutically acceptable excipients onto the inert core of step i) to obtain seal coated tablet;
iii) Coating a dispersion or solution of sucrose and optionally one or more pharmaceutically acceptable excipients onto the seal coated tablet of step ii) to obtain sub coated tablet;
iv) Coating a dispersion or solution of sucrose, binder and optionally one or more pharmaceutically acceptable excipients onto the coated tablet of step iii) to obtain sugar barrier coated tablet;
v) Dissolving/dispersing sirolimus, poloxamer, microcrystalline cellulose, hydroxypropyl methylcellulose and/or hydroxypropyl cellulose and, optionally, one or more pharmaceutically acceptable ingredients in a suitable vehicle;
vi) Coating the dispersion/solution of step v) onto the inert coated tablet of step iv);
vii) Coating a dispersion of antioxidant, sucrose and, optionally, one or more pharmaceutically acceptable excipients in a suitable solvent onto the drug coated tablet of step vi)
viii) Optionally, further film coating the tablet of step vii)

According to another embodiment, there is provided a process for the preparation of sugar over coated tablet, comprising the steps of:
i) Dissolving/dispersing sirolimus, poloxamer, microcrystalline cellulose, hydroxypropyl methylcellulose and/or hydroxypropyl cellulose and, optionally, one or more pharmaceutically acceptable ingredients in a suitable vehicle;
ii) Wet milling the dispersion of step i) into a desired particle size range;
iii) Coating the milled dispersion of sirolimus of step ii) onto an inert core); and
iv) Optionally, coating drug coated core of step iii).

The solvent used for granulation and coating may be selected from water, alcohols like methyl alcohol, ethyl alcohol or isopropyl alcohol, acetone, and mixture thereof.

The invention is further illustrated by the following examples but they should not be construed as limiting the scope of this invention in any way.

### EXAMPLES

### Example 1

| Ingredient | Qty/tab (mg) |
|---|---|
| Inert core tablets | |
| Lactose | 129.00 |
| Polyethylene glycol-6000 | 15.00 |
| Talc | 3.00 |
| Magnesium stearate | 3.00 |
| Seal coating | |
| Pharmaceutical glaze | 9.00 |
| (50% shellac solution) | |
| Talc | q.s |
| Absolute alcohol | q.s to make 25% solution |
| Sub Coating | |
| Sub Coat* | 38.60 |
| Talc | q.s |
| water | q.s |
| Sugar barrier coat | |
| Sucrose | 7.95 |
| Hydroxypropyl methylcellulose | 0.05 |
| Microcrystalline cellulose | 2.00 |
| Purified water | qs |
| Drug layering | |
| Sirolimus (d₉₀₋ 4.2 μm) | 2.00 |
| Poloxamer-407 | 1.00 |
| Hydroxypropyl methylcellulose | 0.50 |
| Microcrystalline cellulose | 2.00 |
| Tocopherol E-acetate | 0.50 |
| Sucrose | 94.00 |
| Purified water | q.s |
| Over coat | |
| Sucrose | 33.00 |
| Hydroxypropyl methylcellulose | 0.17 |
| Tocopherol E-acetate** | 0.50 |
| Water | qs |
| Color Coat | |
| Opalux yellow | 20.00 |
| Water | qs |
| Polishing | |
| Carnauba wax | 1.00 |
| Methanol | qs |

*Sub Coat contains Sucrose-65%, Calcium Sulfate-22%, MCC-8%, Macrogol/PEG-20000-2% and Titanium dioxide-2%.
**contains DL-alpha tocopherol acetate (50%) starch, fish gelatin, sugar, Silicon-dioxide (E 551)

### Procedure:

### A. Preparation of Inert Core

i) Lactose, polyethylene glycol, talc and magnesium stearate were blended together and compressed into a suitable tablet;

### B. Seal Coating

i) Pharmaceutical glaze was diluted to 25% w/w solution using absolute alcohol;
ii) Inert tablets of step A were coated with solution of step i) and during the coating process talc was intermittently sprinkled to prevent sticking of the tablets;

### C. Sub Coating

i) Sub coat was dispersed in water to obtain a 70% w/w of sub coat suspension;
ii) The suspension of step i) was used to coat the coated tablets of step B and during the coating process talc was intermittently sprinkled to prevent sticking of the tablets;

### D. Sugar Barrier Coat

i) Sucrose, microcrystalline cellulose and hydroxypropyl methylcellulose were dispersed in water;
ii) Dispersion of step i) was coated over the coated tablet of step C;

### E. Drug Layering

i) Poloxamer 407 was dissolved in water;
ii) Hydroxypropyl methylcellulose was dissolved in solution of step i);
iii) Sucrose was added in solution of step ii) under stirring;
iv) Microcrystalline cellulose was dispersed in syrup of step iii) under stirring;
v) Sirolimus was dispersed in syrup of step iv) under stirring;
vi) Tocopherol was dispersed in dispersion of step v) under stirring;
vii) The resulting dispersion was coated onto the sugar barrier coated tablet of step D;

### F. Over Coat

i) Sucrose, tocopherol and HPMC were dispersed in water;
ii) Dispersion of step i) was coated over the coated tablet of step E;

### G. Color Coat

i) Opalux yellow was dispersed in water;
ii) Dispersion of step i) was coated onto the over coated tablet of step F;

### H. Polishing

i) Carnauba wax was dispersed in methanol;
ii) Dispersion of step i) was coated over the color coated tablet of step G.

### Bioequivalence Study

Bioavailability study of the Sirolimus tablet (2 mg) tablet of Example 1 was carried out on healthy male volunteers (n=7) taking Rapamune® (2 mg) produced by Wyeth Pharmaceuticals as the reference, the results of which are represented in Table 1 and 2. The objective of this study was to show that a formulation of Example 1 provides an activity and safety profile that is similar to one obtained with an equivalent product in the market.

Single dose (2 mg) two way crossover and open randomized study was designed as, two treatment, two period, two sequence study was used for comparative bioavailability of sirolimus tablet of Example 1 and Rapamune® tablet (2 mg) of Wyeth Pharmaceuticals, under fasting and fed conditions.

TABLE 1
Comparative pharmacokinetic parameters for the sirolimus (Example
2) and Rapamune ® tablet under fed conditions.

| | Cₘₐₓ | AUC₀₋ₜ | AUC_{0-∞} |
|---|---|---|---|
| N = 7 | (ng/ml) | (ng · h/ml) | (ng · h/ml) |
| Sirolimus tablet | 17.4964 | 355.6890 | 420.9476 |
| (Test) | CV %30.4 | CV %46.6 | CV %41.5 |
| Sirolimus tablet | 16.7475 | 348.2036 | 434.1065 |
| (Ref.) | CV %22.9 | CV %32.4 | CV %35.5 |
| Test/Ref. % | 104.90 | 102.56 | 96.68 |
| (90% confidence | (93.29- | (80.77- | (79.92- |
| interval) | 117.95)% | 130.24)% | 116.95)% |

TABLE 2
Comparative pharmacokinetic parameters for the sirolimus (Example
2) and Rapamune ® tablet under fasting conditions.

| | Cₘₐₓ | AUC₀₋ₜ | AUC_{0-∞} |
|---|---|---|---|
| | (ng/ml) | (ng · h/ml) | (ng · h/ml) |
| Sirolimus tablet | 8.9517 | 301.5530 | 381.9564 |
| (Test) | CV %21.6 | CV %32.1 | CV %32.5 |
| Sirolimus tablet | 9.2593 | 290.3494 | 380.4703 |
| (Ref.) | CV %37.1 | CV %25.5 | CV %21.8 |
| Test/Ref. % | 97.67 | 103.66 | 100.81 |
| (90% confidence | (81.88- | (86.45- | (81.67- |
| interval) | 116.50)% | 124.29)% | 124.42)% |

AUC_{0-∞}: for sirolimus tablet was within 80-125% (at 90% Confidence Interval) as shown in Table 1 and 2. The results show that sirolimus 2 mg tablets prepared as per the examples described herein have bioavailability comparable to the reference product, Rapamune® tablet 2 mg of Wyeth Pharmaceuticals, USA.

### Example 2

| Ingredient | Qty/tab (mg) |
|---|---|
| Inert core tablets | |
| Lactose | 129.00 |
| Polyethylene glycol-6000 | 15.00 |
| Talc | 3.00 |
| Magnesium stearate | 3.00 |
| Seal coating | |
| Pharmaceutical glaze | 3.50 |
| (50% shellac solution) | |
| Talc | 1.00 |
| Absolute alcohol | q.s to make 25% solution |
| Sub Coating | |
| Sub Coat* | 38.00 |
| Talc | 0.50 |
| water | q.s |
| Sugar barrier coat | |
| Sucrose | 8.00 |
| Microcrystalline cellulose | 2.00 |
| Purified water | qs |
| Drug layering | |
| Sirolimus | 2.04 |
| Poloxamer-407 | 1.00 |
| Hydroxypropyl methylcellulose | 0.20 |
| Microcrystalline cellulose | 0.20 |
| Vitamin E | 0.25 |
| Sucrose | 96.31 |
| Purified water | q.s |
| Over coat | |
| Sucrose | 36.00 |
| Water | qs |
| Color Coat | |
| Opalux yellow | 2.40 |
| Sucrose | 18.47 |
| Hydroxypropyl methylcellulose | 0.10 |
| Water | qs |
| Polishing | |
| Carnauba wax | 0.03 |
| Methanol | qs |

*Sub Coat contains Sucrose-65%, Calcium Sulfate-22%, MCC-8%, Macrogol/PEG-20000-2% and Titanium dioxide-2%.

### Procedure:

### A. Preparation of Inert Core

i) Lactose, polyethylene glycol, talc and magnesium stearate were blended together and compressed into a suitable tablet;

### B. Seal Coating

i) Pharmaceutical glaze was diluted to 25% w/w solution using absolute alcohol;
ii) Inert tablets of step A were coated with solution of step i) and during the coating process talc was intermittently sprinkled to prevent sticking of the tablets;

### C. Sub Coating

i) Sub coat was dispersed in water to obtain a 70% w/w of sub coat suspension;
ii) The suspension of step i) was used to coat the coated tablets of step B and during the coating process talc was intermittently sprinkled to prevent sticking of the tablets;

### D. Sugar Barrier Coat

i) Sucrose, and MCC were dispersed in water;
ii) Dispersion of step i) was coated over the coated tablet of step C;

### E. Drug Layering

i) Poloxamer 407 was dissolved in part of water;
ii) Sirolimus was dispersed in solution of step i) under stirring;
iii) Hydroxypropyl methylcellulose was dissolved in another part of water;
iv) Vitamin E was loaded over sucrose using low shear mixture;
v) Vitamin E loaded sucrose was dispersed in solution of step iii);
vi) Dispersion of step ii) was added into dispersion of step v);
vii) Microcrystalline cellulose was dispersed in dispersion of step vi) under stirring;
viii) The resulting dispersion (d₉₀-5.46 and d₅₀-2.12) was coated onto the sugar barrier coated tablet of step D;

### F. Over Coat

i) Sucrose was dispersed in water;
ii) Dispersion of step i) was coated over the coated tablet of step E;

### G. Color Coat

i) HPMC and sucrose were dissolved in water;
ii) Opalux yellow was dispersed in solution of step i);
iii) Dispersion of step ii) was coated onto the over coated tablet of step F;

### H. Polishing

i) Carnauba wax was dispersed in methanol
ii) Dispersion of step i) was coated over the color coated tablet of step G.

### Example 3

| Ingredient | Qty/tab (mg) |
|---|---|
| Inert core tablets | |
| Lactose | 129.00 |
| Polyethylene glycol-6000 | 15.00 |
| Talc | 3.00 |
| Magnesium stearate | 3.00 |
| Seal coating | |
| Pharmaceutical glaze | 3.50 |
| (50% shellac solution) | |
| Talc | 1.00 |
| Absolute alcohol | q.s to make 25% solution |
| Sub Coating | |
| *Sub Coat | 38.00 |
| Talc | 0.50 |
| water | q.s |
| Sugar barrier coat | |
| Sucrose | 7.95 |
| Microcrystalline cellulose | 2.00 |
| HPMC | 0.05 |
| Purified water | qs |
| Drug layering | |
| Sirolimus | 2.04 |
| Poloxamer-407 | 1.00 |
| Hydroxypropyl methylcellulose | 0.50 |
| Microcrystalline cellulose | 2.00 |
| Vitamin E | 0.25 |
| Sucrose | 94.25 |
| Purified water | q.s |
| Over coat | |
| Sucrose | 35.82 |
| HPMC-E5 | 0.18 |
| Water | qs |
| Color Coat | |
| Opalux yellow | 2.40 |
| Sucrose | 17.5 |
| Hydroxypropyl methylcellulose | 0.10 |
| Water | qs |
| Polishing | |
| Carnauba wax | 1.0 |
| Methanol | qs |

*Sub Coat contains Sucrose-65%, Calcium Sulfate-22%, MCC-8%, Macrogol/PEG-20000-2% and Titanium dioxide-2%.

### Procedure:

### A. Preparation of Inert Core

i) Lactose, polyethylene glycol, talc and magnesium stearate were blended together and compressed into a suitable tablet;

### B. Seal Coating

i) Pharmaceutical glaze was diluted to 25% w/w solution using absolute alcohol;
ii) Inert tablets of step A were coated with solution of step i) and during the coating process talc was intermittently sprinkled to prevent sticking of the tablets;

### C. Sub Coating

i) Sub coat was dispersed in water to obtain a 70% w/w of sub coat suspension;
ii) The suspension of step i) was used to coat the coated tablets of step B and during the coating process talc was intermittently sprinkled to prevent sticking of the tablets;

### D. Sugar Barrier Coat

i) Sucrose, HPMC and MCC were dispersed in water;
ii) Dispersion of step i) was coated over the coated tablet of step C;

### E. Drug Layering

i) Poloxamer 407 was dissolved in part of water;
ii) Sirolimus was dispersed in solution of step i) under stirring;
iii) Hydroxypropyl methylcellulose was dissolved in another part of water;
iv) Vitamin E was loaded over sucrose using low shear mixture;
v) Vitamin E loaded sucrose was dispersed in solution of step iii);
vi) Dispersion of step ii) was added into dispersion of step v);
vii) Microcrystalline cellulose was dispersed in dispersion of step vi) under stirring;
viii) The resulting dispersion was coated onto the sugar barrier coated tablet of step D;

### F. Over Coat

i) Sucrose and HPMC were dispersed in water;
ii) Dispersion of step i) was coated over the coated tablet of step E;

### G. Color Coat

i) HPMC and sucrose were dissolved in water;
ii) Opalux yellow was dispersed in solution of step i);
iii) Dispersion of step ii) was coated onto the over coated tablet of step F;

### H. Polishing

i) Carnauba wax was dispersed in methanol;
ii) Dispersion of step i) was coated over the color coated tablet of step G.

1. A pharmaceutical composition of sirolimus comprising an inert core and a sugar overcoat, wherein said sugar overcoat comprises
a) sirolimus;
b) poloxamer;
c) microcrystalline cellulose; and
d) a binder selected from the group consisting of hydroxypropylcellulose, hydroxypropyl methyl cellulose or a mixture thereof;
and wherein the poloxamer is not poloxamer 188.

2. The pharmaceutical composition according to claim 1 wherein the poloxamer is present in the amount of about 0.05-5% w/w.

3. The pharmaceutical composition according to claim 1 wherein the poloxamer is selected from the group comprising poloxamer 237, poloxamer 338 or poloxamer 407.

4. The pharmaceutical composition according to claim 1 wherein the binder is present in the amount of from about 0.05% to about 4% w/w.

5. The pharmaceutical composition according to claim 1 wherein the microcrystalline cellulose is present in the amount of from about 0.05% to about 3% w/w.

6. The pharmaceutical composition according to claim 5 wherein the microcrystalline cellulose is present in the amount of from about 0.05% to about 0.3% w/w.

7. The pharmaceutical composition according to claim 1 wherein the sirolimus have particle size with d₉₀ value of from about 2μ to about 10μ.

8. The pharmaceutical composition according to claim 1, which further comprises an antioxidant.

9. The pharmaceutical composition according to claim 1 wherein the pharmaceutical composition is a tablet or a capsule.

10. The pharmaceutical composition according to claim 9 wherein the tablet comprises an inert tablet core and a sugar overcoat.

11. The pharmaceutical composition according to claim 1 wherein the pharmaceutical composition is prepared by a process comprising the steps of:
a) preparation of an inert core;
b) preparation of a dispersion of Sirolimus, poloxamer, microcrystalline cellulose, binder and optionally one or more pharmaceutically acceptable excipients in a suitable vehicle;
c) coating the inert core of step a) with the sirolimus dispersion of step b);
d) optionally, further coating said drug coated cores to obtain the desired pharmaceutical composition.

12. The pharmaceutical composition according to claim 11 wherein the pharmaceutical composition is prepared by a process which further comprises either or both the steps of:
a) seal coating the inert core obtained in step;
b) sub coating the seal coated core.

13. The pharmaceutical composition according to claim 11 wherein the pharmaceutically acceptable excipients include sugars, diluents, lubricant/glidant, disintegrating agent, and coloring agents.
